(19) United States
(12) Patent Application Publication
(10) Pub. No.: US 2025/0001348 A1
(43) Pub. Date: Jan. 2, 2025

(54) DEVICE FOR TREATING AN AIRFLOW

(71) Applicant: BIOTEOS, Willems (FR)

(72) Inventors: Romain BAHEUX, Lille (FR); Romain DHENIN, Rancourt (FR)

(73) Assignee: BIOTEOS, Willems (FR)

(21) Appl. No.: 18/711,469

(22) PCT Filed: Nov. 15, 2022
(86) PCT No.: PCT/EP2022/082008

§ 371 Date: May 17, 2024

(30) Foreign Application Priority Data
Nov. 18, 2021 (FR) FR2112214

(51) Int. Cl. B01D 47/10; A61L 9/20; B01D 45/16; B01D 50/40; B01D 53/84; C12M 1/00; C12M 1/12

(52) U.S. Cl. CPC B01D 47/10; A61L 9/20; B01D 45/16; B01D 50/40; B01D 53/84; C12M 29/24; C12M 31/10; C12M 37/00; A61L 2209/12; B01D 2251/95; B01D 2258/06

(57) ABSTRACT

The invention relates to a device (1) for treating a flow of gas, for example of air, comprising a means (6) for dispersing a liquid in the form of droplets, preferably in the form of microdroplets, into said flow of gas to be treated, and an ultraviolet treatment means configured to subject the flow of gas to be treated to UV radiation. The UV treatment means is positioned downstream of the means (6) for dispersing a liquid in the form of droplets, and the UV treatment means is mounted inside a liquid-gas separation means (10) so as to separate the liquid present in the flow of gas when the flow of gas to be treated is subjected to the UV radiation.

### TECHNICAL FIELD

The present invention relates to the field of gas treatment devices, and more particularly to the field of devices that treat air by injecting liquid into the flow of gas to be treated.

### PRIOR ART

There are gas flow treatment devices. In such devices, as described for example in documents FR 2 452 311 or US 2021/252447, a liquid is introduced into the flow of gas, in the form of droplets, in order to capture the dust and particles present in the flow of gas, and then the liquid, together with the captured dust and particles, is separated from the flow of gas.

More specifically, such devices involve the coating of dusts with liquid and are generally intended for the purification of industrial fumes, which must be freed of harmful dusts before being discharged into the open air.

In such devices, water is sprayed at high pressure into the converging part of a Venturi fed by the gas to be treated. This spray produces very fine droplets whose probability of collision with dust is increased by the turbulence effects generated by the Venturi.

However, such devices, intended for industrial fume treatment in particular, remain cumbersome, and not totally satisfactory in their airflow treatment efficiency.

There are also air treatment devices comprising a sterilization device configured to eliminate pathogenic germs, in particular using ultraviolet (UV) light. Such a device is described in particular in document WO 2021/148211 and comprises a UV module positioned upstream of the introduction of liquid into the flow of gas.

However, as with the previous documents, such a device remains cumbersome, and is not completely satisfactory in terms of airflow treatment efficiency.

There is thus a need for an efficient and compact airflow treatment device, particularly for use in public spaces, especially enclosed ones, such as indoor or underground spaces, where the space available for such a device is limited.

### DISCLOSURE OF THE INVENTION

The present invention aims to solve the various technical problems set out above. In particular, the present invention aims to provide a device for treating a flow of gas, in particular a flow of air, which is compact and efficient. The present invention also aims to propose a gas flow treatment device suitable for treating the air present in a given space, in particular a confined space.

Thus, according to one aspect, the invention relates to a device for treating a flow of gas, for example of air, comprising a means for dispersing a liquid in the form of droplets, preferably in the form of microdroplets, into said flow of gas to be treated, and an ultraviolet treatment means configured to subject the flow of gas to be treated to UV. The UV treatment means is positioned downstream of the means for dispersing a liquid in the form of droplets, and the UV treatment means is mounted inside a liquid-gas separation means so as to separate the liquid present in the flow of gas when the flow of gas to be treated is subjected to the UV radiation.

The liquid-gas separation means, with its integrated UV treatment means, enables two treatment operations to be carried out simultaneously on the flow of gas: firstly, the flow of gas is dissociated from the liquid it contains, notably by centrifugation, and secondly, it is treated by UV radiation to destroy certain pathogenic agents, notably pathogenic germs.

The liquid-gas separation means therefore makes the air treatment device more compact. In addition, the combination of liquid-gas separation may require a relatively long presence time of the flow of gas in the liquid-gas separation means, allowing the UV treatment means to act even more effectively.

Finally, such combined means are used at the end of the process for treating the flow of gas, that is after treatment by dispersing liquid droplets in the flow of gas, and just before releasing the treated flow of gas outside the treatment device. The UV radiation is therefore used to kill only the microorganisms remaining in the flow of gas, and not all those present in the flow of gas entering the treatment device according to the invention.

Preferably, the liquid-gas separation means is configured to separate the liquid contained in the flow of gas by centrifugation, and the UV treatment means is positioned in the middle of the path traveled by the flow of gas to be treated in the liquid-gas separation means.

Centrifugal separation enables the flow of gas with liquid droplets to make a longer journey, during which the liquid and gas are separated. In particular, such an extended path makes it possible to extend the duration of exposure to the UV radiation emitted by the UV treatment means positioned in the middle of such an extended path.

Preferably, the liquid-gas separation means is configured to guide the gas flow to be treated along a helical path, for example along a cylindrical helix or a conical helix, and the UV treatment means is positioned along the axis of said helical path.

The helical path of the gas flow with liquid droplets enables a so-called “cyclone” separation wherein the fluids to be separated are carried along a helical path similar to a vortex, during which the centrifugal force applied to the different fluids present leads to a separation of the fluids in question according to their density. The result is not only efficient separation of the liquid from the gas, but also lengthier exposure of the gas to UV radiation.

Preferably, the liquid-gas separation means is oriented substantially vertically, with the inlet for the flow of gas to be treated at the bottom, and the outlet at the top.

The vertical orientation of the liquid-gas separation means makes it possible for the liquid to be recovered directly and easily from the bottom of the separation means.

Preferably, the liquid dispersion means comprises a Venturi constriction, and a liquid introduction nozzle arranged in or near the Venturi constriction, for example a ring with one or more centripetal liquid introduction orifices, mounted in the Venturi constriction.

The means of dispersing a liquid can be a Venturi constriction, that is a narrowing of the cross-section in which the gas flows, so as to obtain a higher velocity and lower pressure. A liquid can then be injected to form droplets, or even microdroplets, in the flow of gas. In particular, injecting a liquid into the flow of gas enables particles and dust present in the flow of gas to be collected within the liquid. Furthermore, injecting droplets, or even microdroplets, increases the specific contact surface between the liquid and the gas in the flow of gas, and therefore increases the possibility of capturing said particles or dust.

Preferably, the liquid dispersion means is oriented substantially vertically, with the inlet for the flow of gas to be treated at the top, and the outlet at the bottom.

The vertical, downward orientation of the dispersion means, in particular in combination with the vertical, upward orientation of the separation means, enables the flow of gas to be treated to follow a U-shaped path, in the middle of which a single liquid recovery means can be provided. Such a vertical U-shaped path reduces the footprint of the air treatment system, by pooling certain common elements used at different points in the treatment process.

Preferably, the treatment device comprises means for collecting and storing the liquid to be dispersed in the flow of gas to be treated and/or separated from the flow of gas to be treated, the collection and storage means comprising for example a lower part with liquid, in communication with an upper part with air, the collection and storage means, for example the upper part, being positioned downstream of the dispersion means and upstream of the separation means.

Preferably, the airflow path through the treatment device is generally U-shaped, with the collection and storage means in the lower part of the U, and the dispersion means and separation means each forming a branch of the U.

This configuration improves liquid recovery by the collection and storage means, particularly by gravity. In particular, the collection and storage means can recover liquid by gravity from the liquid dispersion means and the liquid-gas separation means.

Preferably, the liquid dispersed in the form of droplets comprises microorganisms, preferably microalgae.

In order to limit the size of the air treatment device, the liquid used to capture the dust and particles present in the flow of gas is the same as the liquid used to treat said particles and dust thus captured. More precisely, the air treatment device directly uses the liquid with microorganisms to supply the means for dispersing a liquid in the flow of gas. Once separated, these droplets of liquid containing microorganisms, dust and particles are then returned to the culture tank where the rest of the liquid with microorganisms is located, in order to treat said dust and particles, with a longer treatment time. There is therefore no dilution of the liquid containing microorganisms by the continuous addition of water droplets. In addition, the means used in the air treatment device and its footprint are optimized, by using the same liquid at different stages of the gas flow treatment process.

Preferably, the liquid dispersed in the form of droplets comprises microalgae, and the collection and storage means comprise microalgae growth means, preferably lighting means.

In order to promote the formation and proliferation of microorganisms, the collection and storage means can include lighting means, preferably arranged vertically and distributed throughout the microorganism growth liquid.

Preferably, the lighting means are positioned substantially vertically in the liquid, and the collection and storage means also comprises bubbling means mounted at a lower end of the lighting means and configured to circulate air bubbles along an outer wall of the lighting means.

In order to limit the formation of biofilm on the surface of the lighting means, which could reduce their effectiveness, and to allow mixing of the microorganisms, the treatment device further comprises bubbling means.

Preferably, the bubbling means comprise nozzles, preferably conical, configured to release a substantially rectilinear flow of bubbles, preferably one behind the other.

In one aspect, a bubbling means is also proposed. The bubbling means comprises means for mounting to a lower end of a lighting means, and is configured to concentrate and release bubbles along the outer wall of the lighting means.

The bubbling means may comprise an air inlet, a bubble-forming means, e.g. a porous material, connected to the air inlet, a frame with in particular a bubble-concentrating means mounted above the bubble-forming means, and the means for mounting to a lighting means positioned above the bubble-concentrating means.

Preferably, the bubble-concentrating means has a frustoconical shape, the lower section of which is configured to receive the bubble-forming means, and the upper section, smaller in size than the lower section, is configured to receive the mounting means.

The bubbling means notably comprises one or more openings, for example above the bubble-concentrating means, notably at the mounting means, enabling bubbles to be released along the lighting means.

In particular, such a bubbling means makes it possible to form a flow of bubbles capable of scraping the outer surface of the lighting means, to remove or prevent biofilm deposits thereon, and to allow mixing of the microorganisms present in the liquid.

### BRIEF DESCRIPTION OF THE FIGURES

FIG. 1 schematically shows a device for treating an airflow according to the present invention;

FIG. 2 shows a cross-sectional side view of a liquid-gas separation means for the treatment device of FIG. 1; and

FIG. 3 shows an exploded perspective view of a bubbling device according to the present invention.

### DESCRIPTION OF THE EMBODIMENTS

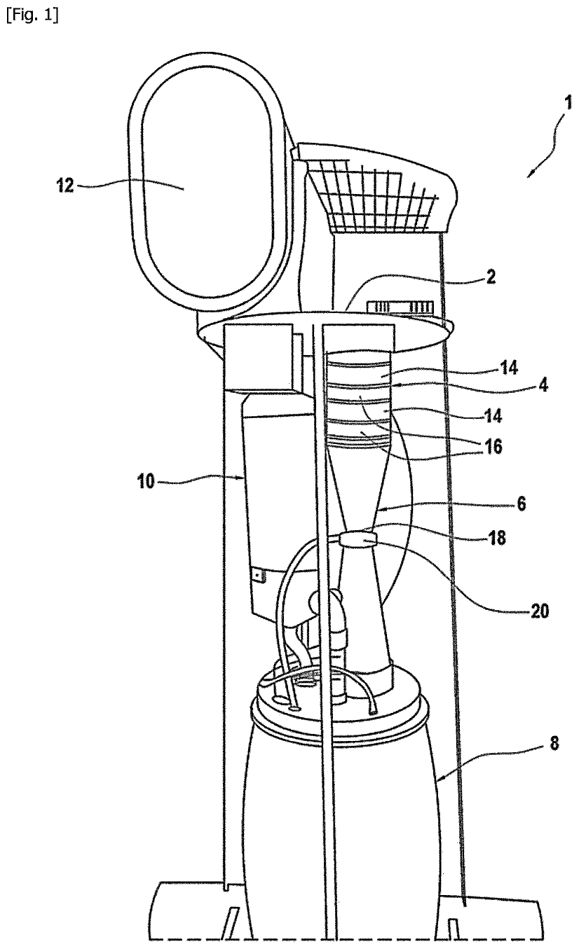

FIG. 1 shows a device 1 for treating a flow of gas, in particular an airflow. The treatment device 1 is designed in particular to treat the ambient air present in a public space, for example an enclosed space, such as the inside of a room or hall, or the inside of a subway station.

In particular, the treatment device 1 must meet purification and space constraints in order to meet the requirements for installation in a public space.

The treatment device 1 thus comprises, preferably in succession according to the airflow in the treatment device 1: an inlet 2 for the air to be treated, airflow entrainment means 4, a liquid dispersion means 6, a collection and storage means 8, a liquid-gas separation means 10 and an air outlet 12.

The entrainment means 4 preferably comprise one or more fans 14, for example two, separated or not by one or more stators 16, for example two. The stators 16 can take the form of fan blades, but with the orientation reversed in relation to that of the fan blades 14. In particular, the stators 16 convert part of the air velocity into static pressure, thus increasing the total pressure of the airflow. The presence of a hexagonal-patterned grille (not shown) at the inlet and outlet of the entrainment means 4 helps guide the air and reduce the turbulence created by the entrainment means 4.

For example, the entrainment means 4 may comprise a hexagonal grille at the output of each stator 16. Such a grille is therefore formed by a plurality of cylindrical channels with a hexagonal base, the axis of which extends in the direction of travel of the airflow. Put another way, the hexagonal-patterned grille has a honeycomb structure.

In addition, and advantageously, the entrainment means 4 may also comprise a free portion (not shown), between the two fans 14, in order to improve the performance of the entrainment means 4 and reduce noise. Such a free portion is advantageously empty, that is devoid of channels and/or blades, so as to let the airflow move freely through it. In particular, this free portion can be used to relax the airflow, creating a homogeneous laminar flow. More precisely, such a free portion is configured to allow the swirling air from the first fan 14 to straighten out again before being drawn in by the second fan 14. Such a free portion thus reduces aeraulic turbulence, which is likely to generate noise in the entrainment means 4. This increases turbine performance by reducing pressure drop and noise.

The free portion is preferably positioned downstream of the hexagonal-patterned grille at the output of the first stator 16, and upstream of the second fan 14. The free portion can have a length, in the direction of airflow, of between 10 mm and 150 mm, preferably between 25 mm and 75 mm. The free portion may, for example, have a length, in the direction of airflow, equal to the sum of the lengths of the first stator 16 and the first hexagonal-patterned grille.

The entrainment means 4 thus create a vacuum at the inlet 2 to draw in ambient air present in the space where the treatment device 1 is installed, and on the other hand to create, with this drawn-in ambient air, an airflow, for example laminar, directed towards the various means of the treatment device 1 which are mounted downstream of the entrainment means 4.

The entrainment means 4 are preferably mounted vertically, with the inlet at the top and the outlet at the bottom.

Thus, the first treatment means through which the airflow created by the entrainment means 4 passes is the liquid dispersion means 6. Preferably, the liquid dispersion means 6 is vertically oriented, that is the airflow follows a substantially vertical trajectory as it passes through the liquid dispersion means 6. The inlet of the liquid dispersion means 6 is at the top, and the outlet is at the bottom, so that the airflow follows a vertical path from top to bottom.

In particular, and as shown in FIG. 1, when the entrainment means 4 are mounted vertically, the airflow is driven vertically, from top to bottom, from the inlet 2 of the treatment device 1 to the outlet of the liquid dispersion means 6, in a substantially rectilinear direction. This limits turbulence and pressure drop in the airflow to be treated.

The liquid dispersion means 6 thus comprises a constriction 18 forming a Venturi, and a liquid introduction nozzle 20.

In a conventional manner, the constriction 18 makes it possible to accelerate the airflow velocity, thus reducing its pressure, through the Venturi effect. An injection nozzle 20 is then provided, in this case at the constriction 18, to obtain an injection of a liquid into the airflow.

More precisely, the injection nozzle 20 can take the form of a ring with dimensions corresponding to the minimum dimensions of the constriction 18, and comprising one or more peripheral openings for injecting a liquid. In other words, the injection nozzle 20 can form the narrowest part of the constriction 18, where the resulting vacuum is greatest. The peripheral opening(s) are preferably oriented perpendicularly to the airflow, that is horizontally in this case, and centripetally, that is towards the center of the ring. The liquid is then injected and dispersed in the form of droplets, or even microdroplets, offering a high contact surface with the air in the airflow to be treated. The liquid droplets thus introduced into the airflow to be treated can effectively capture and coat particles and/or dust present in the airflow, with a view to removing them from the airflow and/or treating them.

The dispersion means 6 may also include a pump for pressurizing the liquid at the injection nozzles, in order to introduce it into the airflow.

At the outlet of dispersion means 6, an airflow is obtained containing liquid droplets trapping dust and/or particles that were present in the airflow to be treated (inlet airflow).

Once the particles and/or dust in the airflow are trapped in the droplets, it is then necessary to extract the droplets from the airflow.

The airflow with the droplets is then conveyed to the collection and storage means 8. The collection and storage means 8 is designed to collect and store droplets dispersed in the airflow, including impurities captured in the airflow. The collection and storage means 8 is thus mounted downstream of the liquid dispersion means 6.

For example, the collection and storage means 8 can be a sealed tank wherein the airflow circulates at the outlet of the liquid dispersion means 6. The inlet of the collection and storage means 8 can be positioned on a horizontal upper face, and communicate directly with the outlet of the liquid dispersion means 6, which provides an airflow in a downward vertical direction.

Similarly, the outlet of the collection and storage means 8 is advantageously positioned on an upper, for example horizontal, face of the collection and storage means 8, in particular to limit the entrainment of droplets with the airflow, towards the outlet of the collection and storage means 8. Thus, in the case of a tank with a top cover, the inlet and outlet can be mounted directly on the cover, preferably at a distance from each other to allow a minimum airflow path in the tank.

The airflow with the droplets is therefore introduced into the collection and storage means 8 through the top, before exiting, also through the top. The droplets present in the airflow are thus destined to fall into the collection and storage means 8, notably under the effect of gravity, when the airflow with droplets circulates in the collection and storage means 8.

Similarly, droplets already separated from the airflow in the liquid dispersing means 6 are also conveyed, by gravity and entrained by the airflow, to the inlet of the collection and storage means 8, which is located below the liquid dispersing means 6.

Similarly, droplets separated from the airflow downstream of the collection and storage means 8 can also be recovered in the latter, notably under the effect of gravity, since the outlet of the collection and storage means 8 is also located below the liquid-gas separation means 10.

In this way, the collection and storage means 8 is configured to collect the droplets dispersed in the airflow. The droplets are stored in the tank at the bottom, with the airflow circulating in the upper part of the tank.

To reduce the size and maintenance requirements of the treatment device 1, the collection and storage means 8 can also be used to supply liquid to the liquid dispersion means 6. More specifically, the liquid injected by nozzle 20 into the airflow can be taken directly from the collection and storage means 8. In this case, there is no need for a water supply, nor for drainage in the event of overflow: The liquid used in the treatment device 1 is used in a closed circuit in the treatment device 1.

To keep maintenance to a minimum, and insofar as it is not possible to prevent droplets from being discharged with the treated airflow at the outlet 12 of the treatment device 1. The collection and storage means 8 can be designed to hold a relatively large volume of liquid, to ensure autonomous and continuous operation of the treatment device 1 for an extended period without intervention. In the case of a tank in particular, it may be planned to fill the tank to 40% of its maximum capacity, or even 60%, 80% or 90%. All you need to do is leave enough space at the top of the tank for the airflow to circulate.

Advantageously, the collection and storage means 8 can include microorganisms, preferably microalgae, configured to treat the dust and/or particles extracted from the airflow by the droplets. Thus, in addition to collecting the particles and dust contained in the airflow, the collection and storage means also enables them to be treated and removed, limiting their concentration in the collection and storage means 8 and further spacing out maintenance operations on the air treatment device. Moreover, the presence of microalgae in the liquid remains compatible with its injection through injection nozzle 20.

The use of microorganisms, and in particular microalgae, is particularly advantageous in the case of the present invention, since a substantial volume of liquid is kept in the collection and storage means 8 to enable operation over an extended period. Particles recovered from the collection and storage means 8 can therefore remain in the collection and storage means 8 for an extended period of time, allowing microorganisms to act on them to destroy them.

In this way, airflow treatment can be combined, combining both the capture of particles by dispersing a liquid in the airflow, and the treatment of said particles by microalgae.

Downstream of collection and storage means 8, treatment device 1 includes liquid-gas separation means 10. The purpose of liquid-gas separation means 10 is to recover the liquid droplets still present in the airflow before it is released to the outside via the outlet 12. The liquid-gas separation means 10 therefore limits the amount of liquid released by the air treatment device 1.

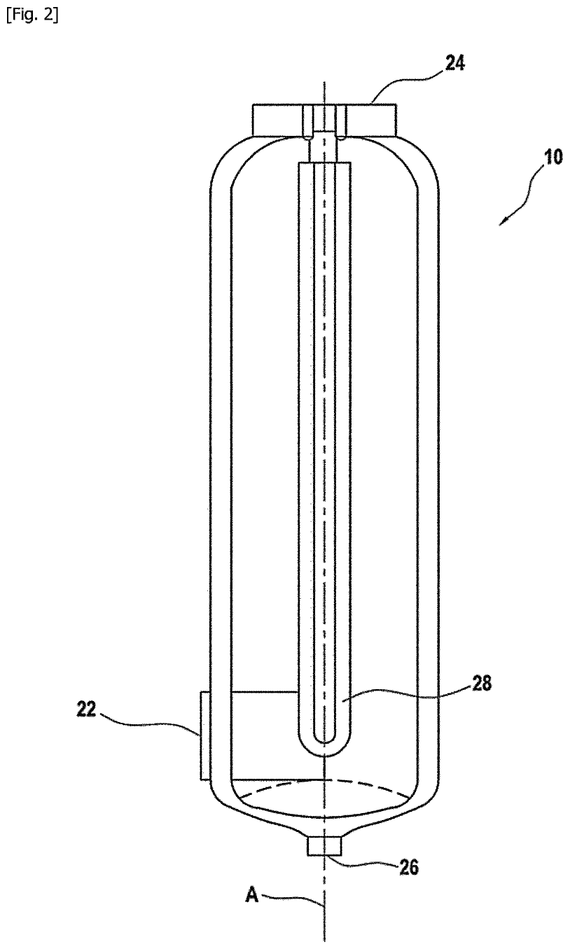

The liquid-gas separation means 10, which is described more precisely in FIG. 2, extends substantially vertically, with an airflow inlet 22 at the bottom, in communication with the outlet of the collection and storage means 8, and an airflow outlet 24 located at the top in communication with the outlet 12 of the treatment device 1. A second outlet 26 for the separated liquid is also provided at the bottom, and is also in communication with the collection and storage means 8.

The liquid-gas separation means 10 is configured to separate the droplets contained in the airflow by centrifugation. Thus, the liquid-gas separation means 10 comprises a generally cylindrical side wall extending along a substantially vertical axis A. The inlet 22 for the airflow with droplets is substantially tangential to the side wall, while the outlets 24 and 26 extend substantially along the axis A.

Thus, when the airflow enters the liquid-gas separation means 10, it takes a helical path along the side wall, that is the airflow takes a path turning along the side wall on the one hand, and rising towards the air outlet 24 on the other. Such a helical path creates centrifugal forces on the different fluids, leading the heavier fluid, in this case the droplets, to be entrained towards the side wall, while the lighter fluid, in this case air, remains closer to the axis A, and can then exit through the outlet 24.

The separated droplets present near or on the side wall are then carried by gravity down the separation means 10, where they are discharged through outlet 26 to the collection and storage means 8 below.

According to the invention, the liquid-gas separation means 10 also includes ultraviolet treatment means 28. The ultraviolet treatment means 28 enables an additional step to be taken in the treatment of the airflow, by destroying any pathogenic germs present in it. The UV treatment means 28, for example a UV lamp, is mounted along the axis A of the liquid-gas separation means 10 and enables UV radiation to be applied to the airflow as it passes through the liquid-gas separation means 10. The inclusion of such a UV treatment means 28 is particularly advantageous, as it does not take up any additional space in the treatment device 1, but is instead fully integrated into the liquid-gas separation means 10. In addition, the positioning of the UV treatment means 28 is made particularly efficient by being able to emit UV radiation onto the airflow for a longer period of time due to the helical path of the airflow in the liquid-gas separation means 10.

Furthermore, as the UV treatment means 28 is mounted downstream of the collection and storage means 8, the UV radiation emitted is used to primarily treat the airflow, and not also the droplets initially dispersed in the airflow. UV radiation is therefore not used unnecessarily, but is instead directed primarily towards airflow treatment.

Once the airflow has been freed of droplets and UV-treated, it exits the liquid-gas separation means 10 via the outlet 24 to join the outlet 12 of the treatment device 1 and be released into the ambient air.

In particular, and as can be seen, the treatment device 1 features a U-shaped circulation path for the airflow to be treated, with the inlet at one of the upper ends of the U, the outlet at the other upper end of the U, the liquid dispersion means 6 and the liquid-gas separation means 10 mounted along the branches of the U, and the collection and storage means 8 in the lower part of the U. Such a configuration is particularly advantageous for limiting the overall dimensions of the treatment device 1, and for improving its operation. Indeed, the positioning of the collection and storage means 8 at the bottom, with the liquid dispersion means 6 and liquid-gas separation means 10 arranged vertically along the arms of the U, enables natural recovery, notably by gravity, of the liquid used to treat the airflow, all along the path of the airflow in the treatment device 1.

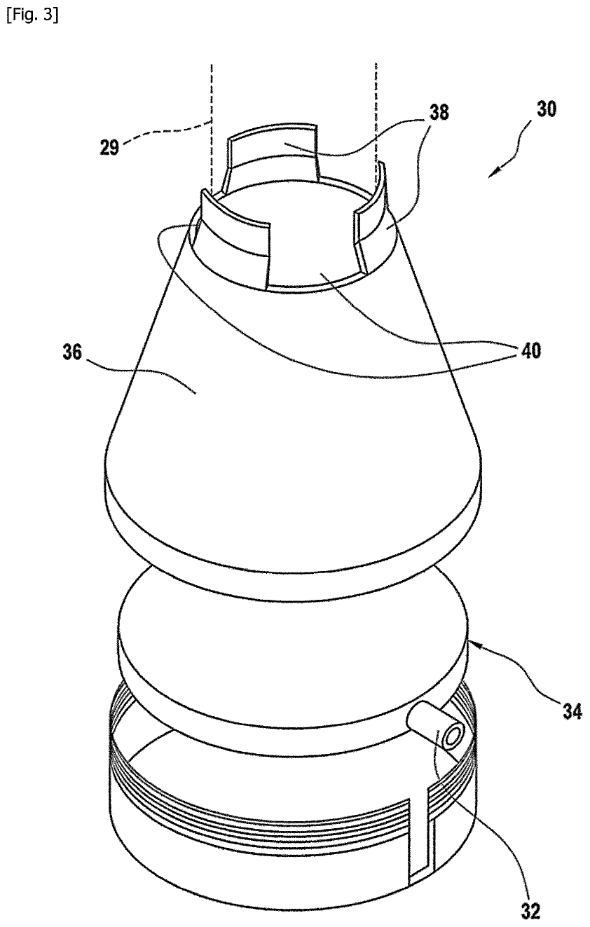

Furthermore, as mentioned above, the liquid used may contain microorganisms, particularly microalgae. To this end, and in order to enable such microalgae to remain and grow in the collection and storage means 8, the latter may comprise lighting means 29 (see FIG. 3). In particular, such lighting means can be arranged vertically, inside the collection and storage means 8, and distributed in the middle of the stored liquid. The lighting means may comprise, for example, one or more LED ribbons, mounted in one or more transparent cylinders immersed in the liquid, in order to supply light peripherally to the microalgae surrounding them.

This type of lighting ensures that the treatment device 1 operates correctly over time, by limiting or spacing out maintenance and servicing operations.

In addition, to maintain a transparent surface of the lighting means, the treatment device 1 may also comprise bubbling means 30 mounted at the lower end of the lighting means 29 and configured to limit deposits along the wall of the lighting means 29. Indeed, such deposits could lead to a reduction in the light transmitted to the microalgae, and therefore to a reduction in their maintenance or grow in the collection and storage medium 8.

The bubbling means 30 comprises an air inlet 32 and a bubble-forming means 34. The bubble-forming means 34 is a porous material, preferably with a large contact surface, which enables a multitude of bubbles released along its upper surface to be produced from the airflow supplied at inlet 32.

The bubbling means 30 also features a bubble-concentrating means 36, for example a frustoconical nozzle mounted between the bubble-forming means 34 and a mounting means 38 of the lighting means 29. More precisely, the frustoconical nozzle has a lower section with a shape and surface area substantially equal to those of the bubble-forming means 34, or even can form a housing for the bubble-forming means 34, and an upper section smaller than the lower section, and with a shape and dimension substantially equal to those of the lighting means 29. The frustoconical nozzle 36 is thus an intermediate piece for connecting the bubble-forming means 34 to the lighting means 29, while also concentrating the bubbles coming from the bubble-forming means 34.

The mounting means 38 holds the lighting means 29 and bubbling means 30 together. The mounting means 38 may, for example, comprise resilient lugs attached to the upper end of the concentration means 36 and resiliently clamping the lighting means 29.

Finally, the bubbling means 30 comprises bubble release openings 40, configured to let bubbles pass through and to release them along the wall of the lighting means 29. In this way, by concentrating and then guiding bubbles along the wall of the lighting means 29, the bubbling means 30 makes it possible to scrape the wall, limiting deposits or removing any deposits present on the surface.

In addition, to supply the bubbling means 30, the treatment device 1 can also use ambient air which, during bubbling, is treated by the liquid wherein it circulates and which, after bubbling, is conveyed with the airflow, to the liquid-gas separation means 10 with the UV treatment means 28, before being released. The bubbling means 30 not only improve the operation of the lighting means 29 over time, but also increase the quantity of air treated by the treatment device 1.

Thus, thanks to the present invention, it becomes possible to effectively treat the ambient air of an enclosed space, with a compact, long-lasting device. In particular, the treatment device according to the present invention makes it possible to combine different types of treatment, while remaining compact and requiring little servicing and maintenance.

1. A device (1) for treating a flow of gas, for example air, comprising a means for dispersing a liquid (6) in the form of droplets, preferably in the form of microdroplets, into said flow of gas to be treated, and an ultraviolet treatment means (28) configured to subject the flow of gas to be treated to UV radiation, characterized in that the UV treatment means (28) is positioned downstream of the means for dispersing a liquid (6) in the form of droplets, and in that the UV treatment means (28) is mounted inside a liquid-gas separation means (10) so as to separate the liquid present in the flow of gas when the flow of gas to be treated is subjected to the UV radiation.

2. The treatment device (1) according to claim 1, wherein the liquid-gas separation means (10) is configured to separate the liquid contained in the flow of gas by centrifugation, and wherein the UV treatment means (28) is positioned in the middle of the path traveled by the flow of gas to be treated in the liquid-gas separation means (10).

3. The treatment device (1) according to claim 1 or 2, wherein the liquid-gas separation means (10) is configured to guide the flow of gas to be treated along a helical path, for example along a cylindrical helix or along a conical helix, and wherein the UV treatment means (28) is positioned along the axis (A) of said helical path.

4. The treatment device (1) according to any of the preceding claims, wherein the liquid-gas separation means (10) is oriented substantially vertically, with the inlet for the flow of gas to be treated at the bottom, and the outlet at the top.

5. The treatment device (1) according to any of the preceding claims, wherein the liquid dispersion means (6) comprises a Venturi constriction (18), and a liquid introduction nozzle (20) arranged in or near the Venturi constriction (18), for example a ring with one or more centripetal liquid introduction orifices, mounted in the Venturi constriction (18).

6. The treatment device (1) according to the preceding claim, wherein the liquid dispersion means (6) is oriented substantially vertically, with the inlet for the flow of gas to be treated at the top and the outlet at the bottom.

7. The treatment device (1) according to any one of the preceding claims, comprising means for collecting and storing (8) the liquid to be dispersed in the flow of gas to be treated and/or separated from the flow of gas to be treated, the collection and storage means (8) being positioned downstream of the dispersion means (6) and upstream of the separation means (10).

8. The treatment device (1) according to any of the preceding claims, wherein the liquid dispersed in droplet form comprises microorganisms, preferably microalgae.

9. The treatment device (1) according to claims 7 and 8, wherein the liquid dispersed in droplet form comprises microalgae, and wherein the collection and storage means (8) comprises microalgae growth means, preferably lighting means (29).

10. The treatment device (1) according to the preceding claim, wherein the lighting means (29) are positioned substantially vertically in the liquid, and wherein the collection and storage means (8) also comprises bubbling means (30) mounted at a lower end of the lighting means (29) and configured to circulate air bubbles along an outer wall of the lighting means (29).

11. The treatment device (1) according to the preceding claim, wherein the bubbling means (30) comprise nozzles (36), preferably conical, configured to release a substantially rectilinear flow of bubbles, preferably one behind the other.
